(19) United States
(12) Patent Application Publication
(10) Pub. No.: US 2011/0182813 A1
(43) Pub. Date: Jul. 28, 2011

(54) AMPHIPHILIC COPOLYMERS AND COMPOSITIONS CONTAINING SUCH POLYMERS

(75) Inventors: Jorge Heller, Ashland, OR (US); Sebastien Jerome Pierre, Maastricht (NL); Mike Gerardus Wilhelmus De Leeuw, Mheer (NL); Jeroen Pieper, Overasselt (NL)

(73) Assignee: NIRVANA'S TREE HOUSE B.V., Maastricht (NL)

(21) Appl. No.: 12/678,811

(22) PCT Filed: Sep. 18, 2008
(86) PCT No.: PCT/EP2008/062441

§ 371 Date: Feb. 16, 2011

(30) Foreign Application Priority Data
Sep. 18, 2007 (EP) 07116651.6

(51) Int. Cl. A61K 38/18; C08G 83/00; A61K 47/34; A61K 38/42; A61K 49/00; A61K 35/12; A61P 43/00

(52) U.S. Cl. 424/9.1; 525/54.1; 514/772.1; 514/13.5; 424/93.7; 514/7.6; 514/8.9; 514/8.8

(57) ABSTRACT

Amphiphilic copolymer, containing at least a hydrophilic chain segment (A) and a hydrophobic chain segment (B), wherein the hydrophilic chain segment (A) contains peptides and wherein the hydrophobic chain segment (B) contains acetal groups or orthoester groups. The hydrophilic chain segment (A) preferably contains glutamine/glutamic acid units or asparagines/aspartic acid units, making a biodegradable copolymer which can form a thermogel.

### FIELD OF THE INVENTION

The invention relates to amphiphilic copolymers, compositions containing the copolymers and at least one therapeutically active agent as well as implants containing the copolymers. The invention also relates to methods of treatment by administering the compositions to a human or animal body.

### BACKGROUND OF THE INVENTION

Controlled release of therapeutically active agents is important in treatments of humans and animals.

In recent years, a number of polymers fabricated into devices as microspheres, microcapsules, liposomes, strands and the like have been developed for this reason. The active agent is incorporated into the interior of the devices and is after administration to the human or animal body slowly released by different mechanisms. U.S. Pat. Nos. 4,079,038, 4,093,709, 4,131,648, 4,138,344, 4,180,646, 4,304,767, 4,946,931 and 5,9689,543 disclose various types of polymers that may be used for the controlled delivery of active agents. The fabrication of such devices is in many cases cumbersome, expensive and may also suffer from irreproducibility in the release kinetics. Furthermore, in most cases an organic solvent is used which may have adverse effect on the therapeutic agent and there could also be residual solvent in the device, which in many cases is highly toxic. Moreover the administration of the solution or dispersion containing the devices is not patient friendly, due to the high viscosity of such solutions or dispersions. Further, such devices are not generally useful for the delivery of proteins that usually undergo a loss of activity during their incorporation into the solid polymer

An important improvement was found in the application of amphiphilic copolymers, containing at least one hydrophilic chain segment (A) and one hydrophobic chain segment (B). Such copolymers may form micelles or thermoreversible gels in water that may contain at least one therapeutically active agent.

Micelles of the amphiphilic copolymer have a number of useful attributes. For example when micelles having the correct size are used, which is usually below 40 nm, they will not extravasate in normal vasculature, but are able to extravasate in a tumor that normally has a leaky vasculature. Because of this it is possible to achieve a high concentration of antineoplastic agents in the tumor, without incurring excessive toxicity in normal tissues.

In addition to the usefulness as micelles in tumor targeting, micelles also find important applications in the solubilization of highly water insoluble drugs, since such drugs may be incorporated in the hydrophobic core of the micelle.

The use of micelles in tumor targeting and solubilization of highly water-insoluble drugs has been extensively described by V. P. Torchilin, Structure and design of polymeric surfactant-based drug delivery systems”, J. Controlled Release 73 (2001) 137-172, and by V. P. Torchilin, “Polymeric Immunomicelles: Carriers of choice for targeted delivery of water-insoluble pharmaceuticals”, Drug Delivery Technology, 4 (20004) 30-39.

Since inflamed tissues also have a leaky vasculature, it is possible to also achieve a high concentration of anti-inflammatory agents in such tissues by incorporating these agents into suitably sized micelles.

Micelles based on poly(ethylene glycol) and poly(D,L-lactic acid) have been investigated by J. Lee, “Incorporation and release behaviour of hydrophobic drug in functionalized poly(D,L-lactide)-block poly(ethylene oxide) micelles” J. Controlled Release, 94 (2004) 323-335. Micelles based on poly(ethylene glycol) and poly(β-benzyl-L-aspartate) have been investigated by Kataoka, G. Kwon, “Block copolymer micelles for drug delivery: loading and release of doxorubicin” J. Controlled Release, 48 (1997) 195-201. Micelles based on poly(ethylene glycol) and poly(ortho ester) have been described by Toncheva et. al., “Use of block copolymers of poly(ortho esters) and poly(ethylene glycol) micellar carriers as potential tumour targeting systems”, J. Drug Targeting, 11 (2003) 345-353.

It is also possible for amphiphilic copolymers having a certain composition to form a so-called thermogel. Such a copolymer has the unique property that at low temperature the copolymers are water soluble, while at higher temperatures the copolymers become insoluble and form a gel. Preferably such copolymers are water soluble at room temperature and at the body temperature of 37° C. they become water-insoluble and form a gel.

The composition containing the copolymer and the therapeutically active agent may be administered at room temperature as a low viscosity solution in water, using a small gauge needle, thus minimizing discomfort for the patient. Once at body temperature the composition will form a well-defined gel that will be localized at the desired site within the body. Further, such materials are also uniquely suited for use with a protein as the therapeutically active agent since the protein is simply dissolved in the same solution that contains the amphiphilic copolymer and the solution is injected, without affecting the properties of the protein.

The ability to use very thin needles makes thermogels well suited for intraocular, and specifically for intravitreal injections. Such injections are of particular interest in the treatment of eye diseases, including age-related macular degeneration (growth of blood vessels inside the vitrous body of the eye).

The therapeutically active agent is slowly released by diffusion, or by a combination of diffusion and erosion, from the micelles or the thermogels made of amphiphilic copolymers. Ultimately, the amphiphilic copolymers has to degrade into fragments that can be metabolized or removed from the body.

Thermogels have been extensively investigated. The most extensively investigated thermogelling polymer is poly(N-isopropyl acrylamide). This polymer is soluble in water below 32° C. and sharply precipitates as the temperature is raised above 32° C. This temperature is known as the lower critical solution temperature, or LCST. Thus, such a polymer could be injected at room temperature as a low viscosity solution using a small bore needle, and once in the tissues, it would precipitate, forming a well-defined depot. However, such polymers are non-degradable. Such polymers were extensively described by Hoffman, in L. C. Dong et. al., “Thermally reversible hydrogels: III. Immobilization of enzymes for feedback reaction control”, J. Controlled Release, 4 (1986) 223-227.

Thermogels using poly(lactide-co-glycolide) copolymers as the hydrophobic segment and poly(ethylene glycol) as the hydrophilic segment have been extensively investigated and are described in a number of patents and publications: U.S. Pat. Nos. 5,702,717, 6,004,573, 6,117,949, 6,201,072 B1. G. Zentner, J. Controlled Release, 72 (2001) 203-215.

Thermogels based on amphiphilic graft copolymers having a hydrophobic poly(lactide-co-glycolide) backbone and poly(ethylene glycol) grafts, or a poly(ethylene glycol) backbone and poly(lactide-co-glycolide) grafts have also been described. Thermogelling polymers with hydrophilic backbones are also known in the art, like for example: PEG-g-PLGA, in Macromolecules, 33 (2000) 8317-8322.

A problem with known amphiphilic copolymers is that the copolymers are not fully degraded and removed from the body, and high molecular weight degradation products, for example poly(ethylene glycol) (PEG) remain in the body and can accumulate inside cells.

In some applications where there is a need for repeated administration of an active agent, such as injections of a thermoreversible gel containing a drug, the use of non-biodegradable materials (including polyethyleneglycol-containing materials) may lead to the formation of large residual molecules. In vascularized tissues these large molecules can often be transported out of the body via blood transport or lymphatic transport. But in areas including the brain, the vitreous body of the eye or intervertebral discs, large molecules cannot escape due to the blood-brain barrier, the blood-eye barrier or fibrous encapsulation. In the absence of biodegradation, these large molecules are likely to accumulate in these tissues, causing toxicity issues or scar formation via encapsulation. The mechanism of degradation of polyethylene glycols and the toxicity issues arising from degradation (degradation occurs with very short polyethylene glycols, smaller than the ones used in biomedical applications) were investigated by Herold et. Al., “Oxidation of polyethylene glycols by alcohol dehydrogenase”, Biochem. Pharmacol., 38 (1989) 73-76. Common polyethylene glycols used for biomedical applications have a molecular weight above 1000 Da, preventing degradation. When they can be transported by blood they usually end up in the liver and the kydneys, as it was shown by Yamaoka et. Al., “Distribution and Tissue Uptake of Poly(ethylene glycol) with Different Molecular Weights after Intravenous Administration to Mice”, J. Pharm. Sci., 83 (1994) 601-606.

The present invention aims at solving these and other problems.

The present invention relates to [claim 1].

### DETAILED DESCRIPTION OF THE INVENTION

The amphiphilic copolymer at the present invention may be a block copolymer or a graft copolymer. In the case of a block copolymer, the copolymer may be an A(BA)ₓ, B(AB)ₓ or (AB)ₓ block copolymer, block A being the hydrophilic chain segment (A), block B being the hydrophobic chain segment (B) and ₓ being an integer between 1 and 5. It is also possible that the amphiphilic copolymer is a graft copolymer where the polymeric backbone of the copolymer is the hydrophilic chain segment (A) or the hydrophobic chain segment (B), and where the polymeric grafted chains are the hydrophobic chain segments (B) when the backbone is hydrophilic, and the hydrophilic segments (A) when the backbone is hydrophobic.

Biodegradation in the context of the present invention refers to the degradation, disassembly or digestion of the amphiphilic copolymers by action of the biological environment, including action of living organisms and most notably at physiological pH and temperature. Preferably the biodegradation takes place in warm-blooded animals and human beings. A principal mechanism for biodegradation in the present invention is the hydrolysis of linkages between and within the monomer units of the amphiphilic copolymers. Specific reactions include for example hydrolysis of acetals, orthoesters, esters and carbonates (chemically or enzymatically), proteolysis of the amide bonds of peptide-based chains and reactions yielding natural-occurring molecules including but not limited to lactic acid, glycolic acid and amino-acids.

The benefit of this invention is that all the polymers degrade in situ into molecules that can be readily transported across the tissue-blood barrier of the eye or the brain, or metabolized by the tissues in situ.

The hydrophilic chain segments (A) contain peptides. Peptides are a sequence of at least 2 amino acids or aminoacid derivatives. Preferably the hydrophilic chain segments contain glutamine/glutamic acid units or asparagine or aspartic acid units. Asparagine is the IUPAC name for aspartamine.

Preferably examples of the glutamine or glutamic acid units have the structure

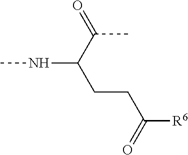

while preferred examples of asparagines or aspartic acid groups have the structure

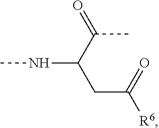

wherein R⁶═OH, OCH₃, NH—(CH₂—)_{z}OH, N—(CH₂—CH₂—OH)₂, NH—(CH₂—CH₂—O—)_{z}H, NH—CH₂—CH(OH)—CH₂—OH, NH—(CH₂—)_{z}O—CO—CH═CH₂, NH—(CH₂—)_{z}O—CO—C(CH₃)═CH₂, N—(CH₃)₂, N—CH—(CH₃)₂.

Preferably the hydrophilic chain segments (A) contain monomeric units of N-(hydroxyalkyl)-L-glutamine, L-glutamic acid, N-(hydroxyalkyl)-L-asparagine, L-aspartic acid, N-alkyl-L-glutamine, N-alkyl-L-asparagine or combinations thereof. More preferably the segment (A) contains monomeric units of N-(2-hydroxyethyl)-L-glutamine or N-isopropyl-L-glutamine (such a segments also being referred to as poly[N-(2-hydroxyethyl)-L-glutamine] or poly[isopropyl-L-glutamine]). Still more preferably the hydrophilic chain segment is poly[N-(hydroxyalkyl)-L-glutamine], poly[L-glutamic acid], poly[N-hydroxyalkyl-L-asparagine], poly[L-aspartic acid], poly[N-alkyl-L-glutamine], poly[N-alkyl-L-asparagine].

Alkyl denotes a hydrocarbyl group having from 1-20 carbon atoms which can be linear, or branched, or a cyclic group having from 3-20 carbon atoms. Examples of alkyl groups include methyl, ethyl, n-propyl, isopropyl, cyclopropyl, n-butyl and t-butyl. By extension, hydroxyalkyl denotes an alkyl chain where one or more carbons have hydroxy-groups attached. Examples of hydroxylalkyl-groups include n-hydroxyethyl-, n-hydroxybutyl-, 1,3-dihydroxy-isopropyl-.

Chain segments containing the monomeric units described above are biodegradable, hydrophilic and they can be prepared in a range of well-defined molecular weights. This enables the fabrication of copolymers that have a well-defined structure, so that well-defined micelles or thermogels can be formed from the copolymers and moreover good reproducibility in the release kinetics of the therapeutically active agent may be achieved.

The hydrophobic chain segments (B) contain acetal groups, ortho ester groups or combinations thereof. Preferred examples of acetal groups have the following structure

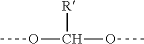

wherein R′═C₁ to C₄ alkyl group (used in formulas I to IV).
Preferred examples of orthoester groups have the structure

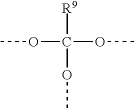

wherein R⁹═H or C₁ to C₄ alkyl groups (used in formulas V to VIII).

Examples of monomers used to make hydrophobic segments containing ortho ester groups include for example those in formula IX as defined below, as well as divinyl ethers like 1-4-cyclohexane dimethanol divinylether, 1-4-butanol divinylether, 1,6-hexanediol divinylether and suitable diols or mixtures of diols. Diols include for example 1-4-cyclohexane dimethanol, 1-4-butanediol, 1,6-hexanediol.

In another preferred embodiment of this invention, acetal and orthoester moieties can be combined in the same hydrophobic segment using both orthoester dienes and divinylethers. Since orthoester dienes react much faster than divinyl ethers with diols, it is preferred to convert an orthoester diene into a diol and then react it with the divinyl ether or vice-versa to limit competition between dienes.

Other hydrophobic segments like for example poly-L-lactide, poly-D,L-lactide, poly(lactide-co-glycolide), polyglycolide, polyanhydride, polyphosphazene, polyketals may also be used.

Combinations of any of those hydrophobic segments may also be used.

Preferred amphiphilic copolymers according to the invention include poly[N-hydroxyalkyl-L-glutamine]-polyacetal, poly[N-hydroxyalkyl-L-glutamine]-polyacetal-poly[N-hydroxyalkyl-L-glutamine], polyacetal-poly[N-hydroxyalkyl-L-glutamine]-polyacetal block copolymers, polyacetal-poly[N-hydroxyalkyl-L-glutamine] graft copolymers, poly[N-hydroxyalkyl-L-glutamine]-poly(ortho ester), poly[N-hydroxyalkyl-L-glutamine]-poly(ortho ester)-poly[N-hydroxyalkyl-L-glutamine], poly(ortho ester)-poly[N-hydroxyalkyl-L-glutamine]-poly(ortho ester) block copolymers, poly(ortho ester)-poly[N-hydroxyalkyl-L-glutamine] graft copolymers, poly[N-hydroxyalkyl-L-asparagine]-polyacetal, poly[N-hydroxyalkyl-L-asparagine]-polyacetal-poly[N-hydroxyalkyl-L-asparagine], polyacetal-poly[N-hydroxyalkyl-L-asparagine]-polyacetal block copolymers, polyacetal-poly[N-hydroxyalkyl-L-asparagine] graft copolymers, poly[N-hydroxyalkyl-L-asparagine]-poly(ortho ester), poly[N-hydroxyalkyl-L-asparagine]-poly(ortho ester)-poly[N-hydroxyalkyl-L-asparagine], poly(ortho ester)-poly[N-hydroxyalkyl-L-asparagine]-poly(ortho ester) block copolymers, poly(ortho ester)-poly[N-hydroxyalkyl-L-asparagine] graft copolymers, poly[N-alkyl-L-glutamine]-polyacetal, poly[N-alkyl-L-glutamine]-polyacetal-poly[N-alkyl-L-glutamine], polyacetal-poly[N-alkyl-L-glutamine]-polyacetal block copolymers, polyacetal-poly[N-alkyl-L-glutamine] graft copolymers, poly[N-alkyl-L-glutamine]-poly(ortho ester), poly[N-alkyl-L-glutamine]-poly(ortho ester)-poly[N-alkyl-L-glutamine], poly(ortho ester)-poly[N-alkyl-L-glutamine]-poly(ortho ester) block copolymers, poly(ortho ester)-poly[N-alkyl-L-glutamine] graft copolymers, poly[N-alkyl-L-asparagine]-polyacetal, poly[N-alkyl-L-asparagine]-polyacetal-poly[N-alkyl-L-asparagine], polyacetal-poly[N-alkyl-L-asparagine]-polyacetal block copolymers, polyacetal-poly[N-alkyl-L-asparagine] graft copolymers, poly[N-alkyl-L-asparagine]-poly(ortho ester), poly[N-alkyl-L-asparagine]-poly(ortho ester)-poly[N-alkyl-L-asparagine], poly(ortho ester)-poly[N-alkyl-L-asparagine]-poly(ortho ester) block copolymers, poly(ortho ester)-poly[N-alkyl-L-asparagine] graft copolymers, poly(L-glutamic acid)-polyacetal, poly(L-glutamic acid)-polyacetal-poly(L-glutamic acid), polyacetal-poly(L-glutamic acid)-polyacetal block copolymers, polyacetal-poly(L-glutamic acid) graft copolymers, poly(L-glutamic acid)-poly(ortho ester), poly(L-glutamic acid)-poly(ortho ester)-poly(L-glutamic acid), poly(ortho ester)-poly(L-glutamic acid)-poly(ortho ester) block copolymers, poly(ortho ester)-poly(L-glutamic acid) graft copolymers, poly(L-aspartic acid)-polyacetal, poly(L-aspartic acid)-polyacetal-poly(L-aspartic acid), polyacetal-poly(L-aspartic acid)-polyacetal block copolymers, polyacetal-poly(L-aspartic acid) graft copolymers, poly(L-aspartic acid)-poly(ortho ester), poly(L-aspartic acid)-poly(ortho ester)-poly(L-aspartic acid), poly(ortho ester)-poly(L-aspartic acid)-poly(ortho ester) block copolymers, poly(ortho ester)-poly(L-aspartic acid) graft copolymers.

The grafted copolymers according to the invention in general have a number average molecular weight between 5000 and 120000 Da, preferably between 10000 and 80000, more preferably between 15000 and 50000. For these grafted copolymers, the hydrophobic backbone preferably has a number average molecular weight between 3000 and 40000 Da. The grafted copolymers may have between for example 3 and 50 hydrophilic side chains with a weight ranging between 500 and 3000 Da (which is around 2 to 20 amino acids derivatives). In general, the grafted copolymers may contain fewer side chains, when the side chains have a higher molecular weight, or a higher number of side chains, when the side chains have a lower molecular weight.

The block copolymers according to the invention may have hydrophilic A blocks having a molecular weight between 300 and 30000 Da, (which would be between 2 and 200 amino acid derivatives) and hydrophobic blocks B having a molecular weight between about 500 and 40000 Da, which would be between 4 and 300 monomers.

Examples of suitable polymers according to the invention are also polymers shown in the formulas I to VIII.

Formula I. Graft copolymer. Copolymer consisting of a random arrangement of the 2 monomeric units figured below. The number of each monomeric unit is an integer between 1 and 300.

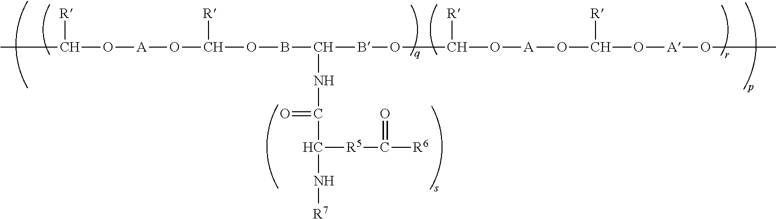

where:

R′ is a C₁ to C₄ alkyl chain.

s=integer from 2 to 20

q and r are independent integers between 1 and 20

p is an integer between 3 and 30

B and B′ are C₁ to C₅ alkyl chains.

A and A′ can be R², R³, or a mixture thereof.

R² is selected from:

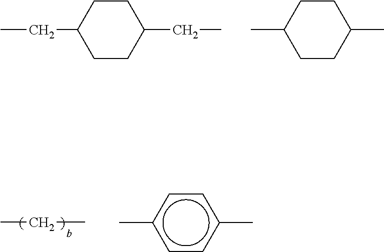

where b is an integer between 1 and 12.

R³ is:

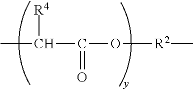

where R⁴ is H or a C₁ to C₆ alkyl chain and y is an integer between 1 and 10.

### R⁵═(CH₂)_{z}

z is integer between 1 and 6.
R⁶═OH, OCH₃, NH—(CH₂—)_{z}OH, N—(CH₂—CH₂—OH)₂, NH—(CH₂—CH₂—O—)_{z}H, NH—CH₂—CH(OH)—CH₂—OH, NH—(CH₂—)_{z}O—CO—CH═CH₂, NH—(CH₂—)_{z}O—CO—C(CH₃)═CH₂, N—(CH₃)₂, N—CH—(CH₃)₂.
z is an integer between 1 and 6.

### R⁷═CO—CH₃, CO—CH═CH₂, CO—C(CH₃)═CH₂.

Preferably R⁵ is a —CH₂— or a —CH₂—CH₂— group.
Formula II. An AB block copolymer.

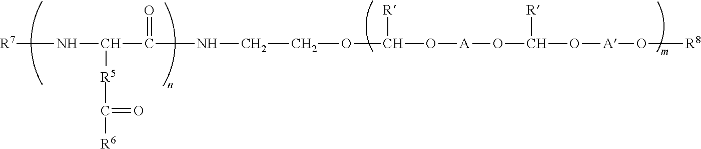

where A, A′, R′, R⁵, R⁶ and R⁷ are defined as stated in formula I;
n is an independent integer between 2 and 200;
m is an independent integer between 2 and 150;

### R⁸═H, R⁷, R¹⁰, CH(R′)—O—R¹⁰.

R¹⁰=C₁ to C₁₆ alkyl chain (linear or branched), cyclohexyl.
Formula III. An ABA block copolymer.

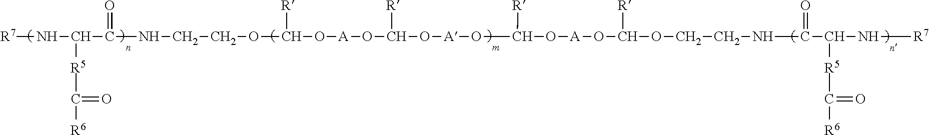

where A, A′, R′, R⁵, R⁶ and R⁷ are defined as stated in formula I;
n and n′ are an independent integers between 2 and 200;
m is an independent integer between 2 and 150;
Formula IV. A BAB block copolymer.

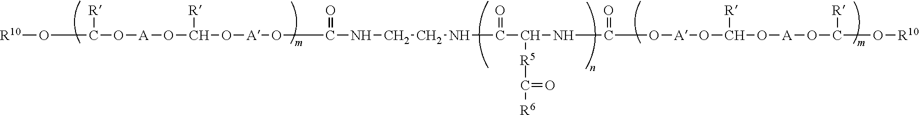

where A, A′, R′, R⁵, R⁶ and R¹⁰ are defined as stated in formula II.
n is an independent integers between 2 and 200;
m is an independent integer between 2 and 150.
Formula V. A graft copolymer. Copolymer consisting of a random arrangement of the 2 monomeric units figured below. The number of each monomeric unit is an integer between 2 and 300.

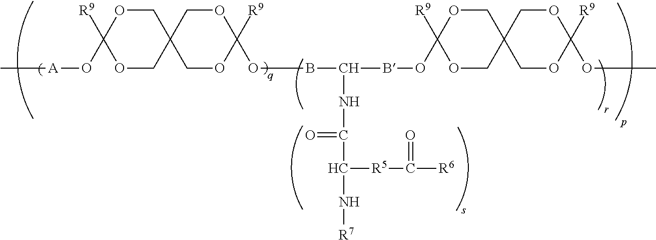

where A, B, B′, R⁵, R⁶ and R⁷ are defined as stated in formula I.
R⁹=—H, C₁ to C₄ alkyl chain.
Formula VI. An AB block copolymer.

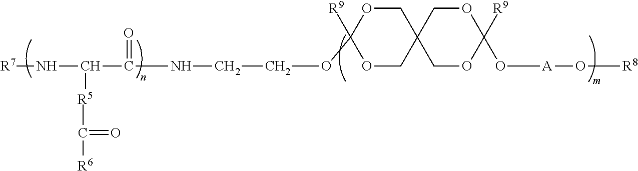

where A, R⁵, R⁶, R⁷ and R⁸ are defined as stated in formula II.
R⁹ is defined as stated in formula V.
n is an independent integers between 2 and 200;
m is an independent integer between 2 and 150;
Formula VII. An ABA block copolymer.

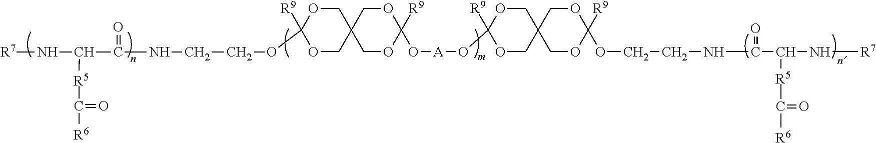

where A, R⁵, R⁶ and R⁷ are defined as stated in formula I.
R⁹ is defined as stated in formula V.
n and n′ are an independent integers between 2 and 200;
m is an independent integer between 2 and 150;
Formula VIII. A BAB block copolymer.

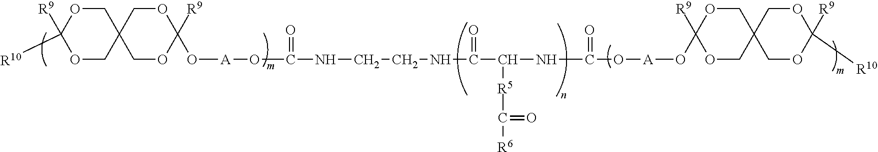

where A, R⁵, R⁶ and R¹⁰ are defined as stated in formula II.
R⁹ is defined as stated in formula V.
n is an independent integer between 2 and 200;
m is an independent integer between 2 and 150;
Formulas IX. Ketene acetal monomeric units for poly(ortho ester) hydrophobic segments (respectively structures 1, 2 and 3).

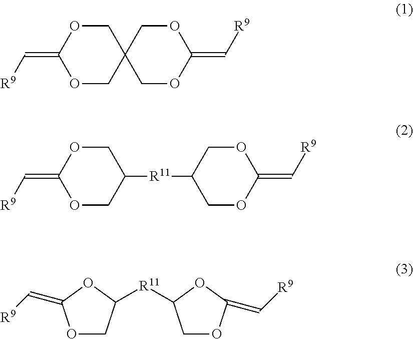

R⁹ is defined as stated in formula V.
R¹¹=—(CH₂)_{d}—, —(CH₂)ₑ—O—(CH₂)_{f}—
d is an integer between 1 and 10, e and f are independent integers between 1 and 6.

The hydrophilic chain segment (A) containing the [N-(2-hydroxyethyl)-L-glutamine] may be prepared by polymerization of suitably substituted N-carboxy anhydrides of a glutamine ester using amino groups on the hydrophobic chain segments to initiate the polymerization. The amino groups in the hydrophobic chain segments may be introduced by incorporating amine-containing alcohols or diols during the polycondensation reaction. By using an amine-containing alcohol like Fmoc-aminoethanol (Fmoc=9H-fluoren-9-ylmethoxycarbonyl)) in the hydrophobic segment polycondensation, polymer with protected-amine endgroups can be synthesized and lead to the formation of amphiphilic block copolymers like the ones in formulas II, III, VI and VII. When using Fmoc-serinol as a monomer for polycondensation, hydrophobic chain segments with amino groups along the polymer chain are formed and can be used to make amphiphilic graft copolymers like the ones in formulas I and IV. Other amine-containing alcohols and diols can be used by people skilled in the art.

In the final synthesis step, aminolysis of the protected sidechain of glutamine by an amine like 2-aminoethanol yields [N-(2-hydroxyethyl)-L-glutamine]. The aminolysis step can use other amines including but not limited to 4-aminobutanol, N-isopropylamine to yield [N-(4-hydroxybutyl)-L-glutamine] or [N-isopropyl-L-glutamine].

Glutamine can be replaced by asparagine to prepare hydrophilic chain segments containing [N-(2-hydroxyethyl)-L-asparagine] or other derivatives of L-asparagine.

In another embodiment, a spacer may be inserted between the amino groups of the hydrophobic segment and the hydrophilic segments to put the amino groups further from the hydrophobic segment polymer chain. Spacers include for example natural and unnatural amino-acids, n-amino-alcanoïc acid (C₂ to C₁₆), and their acid halide and anhydride derivatives. Hetero-bifunctional polyethylene glycols terminated with an amino group and a carboxylic acid group are available as well (1 to 8 ethylene glycols units). The amine moieties of the spacer may be protected to react it with the hydrophobic chain segment and then deprotected prior to the hydrophilic segment formation.

An alternative way to synthesize the hydrophilic chain segment is to use molecular biology to produce the glutamine or asparagine polypeptide using a living organism, including for example yeasts and bacteria.

Hydrophobic chain segments (B) that contain acetal groups may be prepared by a transacetalization reaction that is an equilibrium reaction and must be driven to high molecular weight by removing the low molecular weight by-products, usually alcohols. Preferably these chain segments are prepared by the reaction of a polyol and a divinyl ether as described by J. Heller et. al., “Preparation of polyacetals by the reaction of divinyl ethers and polyols” J. Polymer Sci., Polymer Letters Ed., 18 (1980) 293-297 and in U.S. Pat. No. 4,713,441.

Chain segments containing the ortho ester groups may be prepared by the addition of polyols to the diketene acetal 3,9-diethylidene-2,4,8,10-tetraoxaspiro[5.5] undecane (DETOSU, see Structure 1 on FIG. 9). Their preparation and applications have been extensively reviewed by Heller, Poly (Ortho Esters), Advances in Polymer Science, Vol. 107, (1993) 41-92 and Heller, et. al., Poly(ortho esters): Synthesis, characterization, properties and uses, Adv. Drug Delivery Rev., 54 (2002) 1015-1039.

Chain segments containing the ortho ester group can also be prepared using Structures 2 and 3 of Formula IX. Structure 2 can be prepared as described by Newsome et. al., U.S. Pat. No. 6,863,782. Structure 3 can be prepared as described by Crivello et. al., Ketene acetal monomers: synthesis and characterization, J. Polymer Sci., Part A: Polymer Chem., 34 (1996) 3091-3102.

It is known by people skilled in the art that chainstoppers can be used to control the final molecular weight of the hydrophobic chain segments by being able to terminate the growth of polymer chains. In this invention, molecules used as chainstoppers can be for example mono-alkenes, preferably monovinyl ethers, or mono-alcohols. Other categories of chainstoppers for the hydrophobic chain segments of this invention include acid halides, anhydrides and activated esters. Most preferably, alcohols with low toxicity such as isopropyl alcohol, diethyleneglycol monoethyl ether or diethyleneglycol monobutyl ether are efficient chainstoppers.

The chain segment containing N-(2-hydroxyethyl)-L-glutamine may be incorporated into the amphiphilic block copolymers by two distinctly different procedures. In one procedure, a hydrophobic, amine-terminated block is prepared, preferably a polyacetal, a poly(ortho ester), or a combination of the two groups with the two terminal amino groups being used to initiate the polymerization of a suitably substituted N-carboxyanhydride. Graft copolymers with a hydrophobic backbone may be prepared using a polyacetal, a poly(ortho ester) or a combination of the two groups with pendant amino groups and the polymerization of a suitably substituted N-carboxyanhydride initiated by the pendant amino groups.

In a second procedure, block or graft copolymers are formed by coupling a suitably monosubstituted poly[N-(2-hydroxyethyl)-L-glutamine] to a polymer, preferably a polyacetal, a poly(ortho ester), or a combination of the two groups containing amino end-groups, or pendant amino groups. Of these two procedures, the initiation of a suitably substituted N-carboxyanhydride by terminal, or pendant amino groups of a polyacetal, a poly(ortho ester) or a combination of the two groups, is preferred since the difficult removal of unreacted poly[N-(2-hydroxyethyl)-L-glutamine] segments is not required.

In a third procedure, an hydrophilic segment such as poly[N-(2-hydroxyethyl)-L-glutamine] may be formed from a N-carboxyanhydride ester of L-glutamic acid using an initiator containing an amine and carboxylic acid. The resulting carboxylic-terminated hydrophilic segment can be reacted with an amine-containing preformed hydrophobic segment to yield an amphiphilic copolymer (block or graft). A method to prepare poly[N-(2-hydroxyethyl)-L-glutamine] terminated with carboxylic acid moiety is described by Schacht et. al. in U.S. Pat. No. 7,005,123 B1.

The copolymers of this invention will find utility in any of the uses for which biodegradable polymers are useful, including such uses as vehicles for the sustained release of therapeutically active agents, orthopedic implants, degradable sutures, and the like, they will also find particular utility in applications where their nature as block and graft copolymers having both hydrophilic and hydrophobic segments confers a special benefit, and those uses will be addressed in greater detail below.

For some applications special moieties may have to be introduced into the polymer chains that allow chemical reactions to occur between the polymer chains to achieve polymer crosslinking. Crosslinking is usually carried out in order to modify the mechanical properties and degradation profile of polymers. There are a number of ways known to those skilled in the art to introduce these moieties into the polymers described above via terminal hydroxy- and amino-groups, known as functionalisation of the main chain and/or sidechains (see R⁶, R⁷ and R⁸ moieties in the formulas I to VIII for examples). These moieties may include, for example, acrylates, methacrylates, vinyl groups, styryl groups, acrylamides, methacrylamides, thiols and thiol-ene dual systems. The activation and intermolecular reaction of these moieties is usually caused by a radiation source, an external chemical reaction or stimulus, or a combination thereof. Radiation examples include, heat, infrared sources, ultra-violet sources, electron-beam sources, micro-waves sources, x-ray sources, visible light sources [monochromatic or not] and gamma-rays. External reaction, or stimulus include, for example, pH, oxidation/reduction reactions, reactions with a chemical agent present in vivo (gas, protein, enzymes, antibody etc), reaction with a chemical added to the composition upon introduction into the body, known as dual systems, for example a molecule containing two or more reactive groups.

The invention also relates to compositions containing at least one amphiphilic copolymer of the present invention and at least one therapeutically active agent.

By therapeutically active agents people skilled in the art refer to any set of molecules, cells or cell materials able to prevent, slow down or cure a disease. Therapeutically active agents include proteins, enzymes, peptides, nucleic acid sequences such as DNA and RNA, complexes of synthetic gene vectors (polyplexes), antigens, antibodies, toxins, viruses, virus-based materials, cells, cell substructures, synthetic drugs, natural drugs and substances derived from these.

Examples of active agents and their pharmaceutical acceptable salts are pharmaceutical, agricultural or cosmetic agents. Suitable pharmaceutical agents include locally or systemically acting pharmaceutically active agents which may be administered to a subject by topical or intralesional application (including, for example, applying to abraded skin, lacerations, puncture wounds etc. . . . , as well as surgical incisions) or by injection, such as subcutaneous, intradermal, intramuscular, intraocular or intra-articular injection. Examples of these agents include, for example, anti-infectives (including antibiotics, antivirals, fungicides, scabicides or pediculicides), antiseptics (e.g., benzalkonium chloride, benzethonium chloride, chlorhexidine gluconate, mafenide acetate, methylbenzethonium chloride, nitrofurazone, nitromersol and the like), steroids (e.g., estrogens, progestins, androgens, adrenocorticoids and the like), therapeutic polypeptides (e.g., insulin, erythropoietin, morphogenic proteins such as bone morphogenic proteins, and the like), analgesics and anti-inflammatory agents (e.g., aspirin, ibuprofen, naproxen, ketorolac, COX-1 inhibitors, COX-2 inhibitors and the like), cancer chemotherapeutic agents (e.g., mechlorethamine, cyclophosphamide, fluorouracil, thioguanine, carmustine, lomustine, melphalan, chlorambucil, streptozocin, methotrexate, vincristine, bleomycin, vinblastine, vindesine, dactinomycine, daunorubicin, doxorubicin, tamoxifen, and the like), narcotics (e.g., morphine, meperidine, codeine and the like), local anesthetics (e.g., amide- or anilide-type local anethestics such as bupivacaine, dibucaine, mepivacaine, procaine, lidocaine, tetracaine and the like), antiemetic agents (e.g., ondansetron, granisetron, tropisetron, metoclopramide, domperidone, scopolamide and the like), antiangiogenic agents (e.g., combrestatine, contortrostatin, anti-VGF and the like), polysaccharides, vaccines, antisense oligonucleotides.

The present invention may also be applied to other locally acting active agents, such as astringents, antiperspirants, irritants, rubefacients, vesicants, sclerosing agents, caustics, escharotics, keratolytic agents, sunscreens and a variety of dermatologics including hypopigmenting and antipruritic agents. The term active agents further includes biocides such as fungicides, pesticides and herbicides, plant growth promoters or inhibitors, preservatives, disinfectants, air purifiers and nutrients. Pro-drugs of the active agents above are included in the scope of this invention.

### Micellar Systems.

In one embodiment of the invention the composition contains micelles of the copolymer with the therapeutically active agent(s) being entrapped in the micelles.

When the copolymers are placed in water, in which the hydrophilic segment is soluble and the hydrophobic segment is insoluble, the polymer chains may spontaneously self-aggregate to form micellar structures depending on their concentration.

One major utility of such micellar structures resides in their ability to entrap and solubilize hydrophobic drugs in the hydrophobic core. Such entrapment can be carried out in a number of ways. The drug may be added to the aqueous media containing the micelles and incorporated by simple stirring, by heating to moderate temperatures or by ultrasonification. Alternately, a drug dissolved in a volatile organic solvent is added to an aqueous solution of preformed micelles with a subsequent solvent evaporation from the system.

Efficient entrapment of hydrophobic drugs requires a highly hydrophobic core. The high hydrophobicity of polyacetals, poly(ortho esters) or their copolymers allows the preparation of micellar systems with significantly enhanced entrapment efficiency relative to other biodegradable segments such as poly(lactic-co-glycolic) acids copolymers.

While any of the anticancer agents that can be incorporated in micellar structures are suitable for this use, anticancer agents that are particularly suitable for micellar tumor targeting are those with low water solubility such as doxorubicin, daunorubicin, epirubicin, mitomicin C, paclitaxel, cis-platin, carboplatin, and the like.

Other agents may include anticancer proteins such as neocarzinostatin, L-aspariginase, and the like and photosensitizers used in photodynamic therapy. In addition to the usefulness as micelles in tumor targeting, micelles also find important applications in the solubilization of highly water insoluble drugs, since such drugs may be incorporated in the hydrophobic core of the micelle.

While any of the anti-inflammatory agents can be incorporated in micellar structures, non-steroidal anti-inflammatory agents of particular interest are meloxicam, piroxicam, piketoprophen, propylphenazone and the like.

### Polymersomes.

In another embodiment of the invention, the composition of the invention contains polymersomes of the copolymer with the therapeutic agent(s) being entrapped in the polymersomes. These are microscopic vesicles of approximately 5 to 10 microns in diameter that consist of an aqueous core surrounded by a thin, yet robust shell formed from the self-assembly of amphiphilic block copolymer or graft copolymer.

One major utility of such polymersomes is artificial blood consisting of haemoglobin contained within polymersomes. Other therapeutic agents can also be used. A review on polymersomes was written by D. Discher “Polymersomes” Annual Review of Biomedical Engineering, 8 (2006) 323-341.

### Thermogels.

In still another embodiment of the invention, a copolymer composition contains the copolymer according to the invention and the therapeutically active agent as a solution in water, the solution having a lower critical solution temperature (LCST) below 37° C.

Such polymers are water-soluble below their LCST, due to strong hydrogen bonding between the hydrophilic part of the chains and water, but above the LCST value hydrogen interactions are weakened and hydrophobic interactions between the hydrophobic domains of the polymer become dominant with consequent phase separation of the polymer resulting in an increase in viscosity, and depending on the polymer concentration, gelation of the solution at higher concentrations.

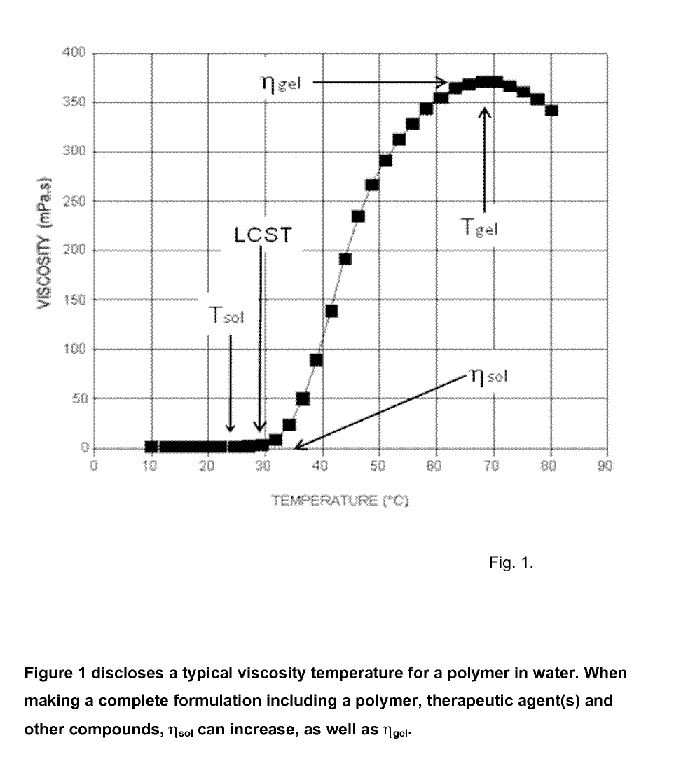

The LCST value depends on the balance of hydrophilic and hydrophobic portions of the block, or graft copolymer and can be adjusted by varying this balance. It also depends on the concentration of the block, or graft copolymer in aqueous solution. Materials having particular usefulness for therapeutic applications are those where the LCST value is between 22 and 37° C. since such materials will be soluble in aqueous solution at room temperature and form a gel at the body temperature of 37° C. The compositions according to the present invention have a Tₛₒₗ, ηₛₒₗ, T_{gel} and η_{gel} as is illustrated in FIG. 1. Tₛₒₗ is a temperature where the formulation is a liquid having a viscosity ηₛₒₗ. After an increase of temperature to a value above the LCST, the composition will start to gel (see FIG. 1). At the T_{gel} or the temperature when the formulation is gelled, the viscosity η_{gel} has increased to a certain plateau viscosity. Tₛₒₗ generally ranges between 0 and 80° C., preferably between 0 and 60° C., most preferably between 4 and 30° C. θₛₒₗ typically is below 500 mPa-s, preferably ≦400 mPa-s, most preferably 300 mPa-s. T_{gel} ranges typically between 10 and 90° C. and is always higher than the Tₛₒₗ of the composition. Typically T_{gel} is 1-20° C. higher then Tₛₒₗ, in a preferred case between 1 and 10° C., most preferred between 1 and 5° C. higher than Tₛₒₗ.

Thermogelation and thermal reversible behavior is obtained when:
η_{gel}/ηₛₒₗ≦2, preferably η_{gel}/ηₛₒₗ≦4, most preferably η_{gel}/ηₛₒₗ≦10

One of the desirable features of thermogels is the ability to administer thermogel formulations using a small bore needle resulting in significantly less pain on administration relative to the administration of microspheres, microcapsules, strands, or other solid drug-releasing devices. This is due to the water solubility of thermogels at room temperature, and the relatively low viscosity of the aqueous solution making the use of small-bore needles possible.

Another important and unique feature is the ability to deliver therapeutically active agents at a controlled rate and without loss of biological activity. In this application, the polymer according to the invention is dissolved in an appropriate volume of water and the peptide, protein or nucleic acid sequence is dissolved in the same solution. The mixture is then injected in the desired body site, where it gels, entrapping the peptide, protein or nucleic acid sequence in the gelled material. It will be appreciated that these are extremely mild conditions since active agents are only exposed to water and temperatures no higher than the body temperature of 37° C.

This method is greatly superior to conventional methods of protein incorporation into solid polymers that require harsh conditions such as elevated temperatures, and/or organic solvents, or mixtures of organic solvents and water that usually results in loss of protein activity.

This method is particularly useful for the delivery and dosing of therapeutically active agents in applications including but not limited to injections of the thermogels containing the proteins mentioned above into articulate cartilage, pericardium, cardiac muscles, sclera and the vitreous body of the eye.

The temperature responsive behaviour also gives advantages when building composite devices. They can be built by using several thermogels with different LCST (always below 37° C.). Upon implantation the in vitro degradation and release of actives can be tuned depending on their LCST and chemical structures.

In a further preferred embodiment the therapeutically active agent is a growth factor. Such a composition is very suitable for use in the treatment of diseases of the intervertebral disc. This is because the composition will gel and hold the active agent in place over a period in time, releasing it in a slower manner than straight injection of a non-gelling solution. Further the gel-forming polymers will be completely broken down after having completed their function. This is especially important in the area of intervertebral discs, where there is less metabolic action.

Preferably as growth factor at least one compound is used of the group consisting of transforming growth factor beta-3, osteogenic protein 1, bone morphogenic protein 2 and 7. Although less preferred it is also possible to use compositions containing thermogels in general and a transforming growth factor. Such a composition at least has the advantage of the slow release of the growth factor.

Another desirable feature of thermogels is the ability to deliver these gels as an aerosol. Advantages of such delivery systems include ease of use and a fast gelling process as a result of its high surface area and its homogenous delivery. In addition, the aerosol ensures intimate contact between the gel-tissue interface. Such a spray delivery system is useful in applications like tissue sealant, artificial skin, anti-adhesion barrier, occlusive wound dressing and for the treatment of chronic wounds like diabetic ulcers.

Another desirable application is the delivery of thermogels to targeted areas using tubular devices such as, but not limited to, catheter or cannula. The catheters can be long (ie 100 cm or longer) or short (ie 20 cm). The gel is initially transferred in liquid form or in pre-gel form from a container under pressure, into the lumen of the catheter or cannula. The liquid or pre-gel is thus lead to the targeted tissue or area of placement inside the body. The tubular device can be used to apply the gel via the vascular system, the lymphatic system. In another embodiment the tubular devices can be used to deliver the thermogels via natural openings in the body like the ear, the nose, the throat, the intestinal tract, the urinary tract and the vagina, in order to reach for instance the sinuses, the middle ear, the inner ear, the stomach, the uterus, the bladder, sections of the gut etc, in order to deliver therapeutic agents such as but not limited to antibiotics, anti-inflammatory agents, analgesics or imaging agents.

During use, tubular devices may warm up to body temperature once placed inside the body, and this could lead to premature gelation of the composition which is flowing through the lumen of the tubular devices, thus blocking or hindering the flow. To avoid this, the tubular devices may be cooled down prior to use or equipped with an additional lumen that can be used to keep the inner lumen below the gelation temperature of the composition.

Bioerodible Copolymer. Matrix for Controlled Delivery, Tissue Engineering and Biomedical Applications.

The invention also relates to an implant containing the polymer according to the invention. In certain uses it is desirable to have a material that has improved mechanical properties relative to thermogelling materials. To this effect, solid polymers can be prepared that are useful in a number of applications, for example orthopaedic applications such as fracture fixation, or repair of osteochondral defects and the like. The solid polymer can be readily fabricated into a number of shapes and forms for implantation, insertion or placement on the body or into body cavities or passageways. For example, the block, or graft copolymer may be injection molded, extruded or compression molded into a thin film, or made into devices of various geometric shapes or forms such as flat, square, round, cylindrical, tubular, discs, rings and the like. Rod, or pellet-shaped devices may be implanted using a trocar, and these, or other shapes, may be implanted by minor surgical procedures. Alternatively, a device may be implanted following a major surgical procedure such as tumor removal in the surgical treatment of cancer. The implantation of polymer wafers containing anticancer agents is described for example, in Brem et. al., U.S. Pat. Nos. 5,626,862 and 5,651,986 and references cited therein; and the block and graft copolymers will find utility in such applications.

Tissue engineering applications using thermogels made with copolymers described in this invention include for example nerve growth or repair, cartilage growth or repair, bone growth or repair, kidney repair, muscle growth or repair, skin growth or repair, secreting gland repair, ophthalmic repair and intervertebral repair. In these applications the thermogels may be combined with cells like nerve-derived cells, chondrocytes, osteoblasts, bone marrow derived stem cells, mesenchymal stem cells, kidney derived cells, muscle derived cells, fibroblasts, keratinocytes, epithelial cells, fat-derived cells, nucleus pulposus cells and annulus fibrosus cells.

It should be underlined that thermogels may be used as such or as a part of a bigger implant, membrane, scaffold or structure. Another desirable application, for example, is using the gel with entrapped therapeutically actives as filler of the lumen of implantable devices that are used as drug delivery container, such as, but not limited to, the OphtaCoil described by Pijls et al “In vivo tolerance and kinetics of a novel ocualr drug delivery device” J Controlled Release, 116 (2006) 47-49. The device can have any shape with a lumen, a partly closed encasing, grooves or profiles that will hold the viscous gel in place. The device containing the gel and therapeutically actives can be used to release various drugs in for example, but not limited to, the eye, ear, buccal cavity, sinuses, gut and intestines, urinary tracts, vagina and uterus and any other organ or location where the device is placed inside the body. The device can be filled with the gel. The polymer composition used needs to gel at lower temperature such as 20-25° C. so that it will not run out of the container prior to implantation. When the devise is implanted, the gel inside will degrade as described above, thereby releasing the drugs entrapped inside the gel. The benefit of using the gel system described above is that the release rate is more constant than when using diffusion systems, and that all the degradation products are biodegradable and biocompatible. Further benefits of using the said gels containing the therapeutic actives as filler of said devices is the ability of the gels to safely entrap the therapeutic agent(s) without degradation or denaturation over the duration of delivery.

Thermogels with LCST below 37° C. may also be used as temporary void fillers in case of significant trauma, to prevent adhesion of damage tissues and scar tissue formation while waiting for corrective and reconstructive surgery. Void filling could be performed easily by injecting the thermogels and removal could be performed via cutting, scraping or suction after cooling down the area to liquefy the thermogel. Other benefits of using voidfillers may include for example: preventing contamination from outside, preventing infection, preventing surrounding tissue necrosis or alteration, inducing specific tissue formation (bone, cartilage, muscle, nerve, skin etc.), helping to maintain structural integrity of the surrounding tissues by itself or by combination with other known scaffolds or structures, trapping specific natural or foreign molecules. The temporary void fillers may be further improved by combining the thermogels with synthetic or natural polymers. These polymers may be present as micro or nano particles, microspheres, powders, fibers, fleeces, membranes, films or combinations thereof. Examples of these polymers include for example polylactide poly(lactic-co-glycolic acid), polycaprolactone, poly trimethylcarbonate, calcium phosphates like tri-calcium phosphate and hydroxyapatite, collagen, gelatin, hyaluronic acid, chondrointin sulfate, chitosan and combinations thereof. Beneficial aspects of adding such polymers to thermogels include improved bulking and void filling capacity, implant containment, tissue inducing capacity, osteoconductivity, tissue compatibility, mechanical properties and improved tailoring in implant resorbtion.

Thermogelling polymers of this invention also exhibit little or no swelling upon gelation when reaching the LCST. This is useful for some implant or tissue-engineering applications such as intra-ocular and intra-cranial surgery, where swelling after implantation can increase pressure for example on nerves, organs or bones and thus can cause damage. The difficulty to find proper materials for orbital reconstruction is described by G. Enislidis “Treatment of orbital fractures: the case for treatment with bioresorbable materials” J. Oral Maxillofacial Surgery, 62 (2004) 869-872.

Another useful utility of thermogels with LCST below 37° C. is in the prevention of post-surgical adhesions. Adhesions are fibrous bands of tissue that form between separate tissues or organs as a result of surgery, trauma or infection. High incidences of adhesions following surgery have been reported in the peritoneal area by Yeo et al. “Polymers in the prevention of peritoneal adhesions” Eur J Pharm and Biopharm, 68 (2008) 57-66. The consequences can be debilitating and may include pain, tissue compression, infertility, inflammation or bowel obstruction. Next to peritoneal adhesions, anti-adhesion barriers can also be applied in neurosurgery for the prevention of arachnoidal and epidural adhesions. Reconstruction of the dura mater, the though fibrous protective membrane which encases the brain and spinal cord, is also of interest. Following a neurosurgical procedure, an appropriate tight seal of the dura needs to be achieved to prevent leakage of the cerebrospinal fluid from the tissues of the brain and spinal cord while fibrous tissue during healing needs to be minimized.

Spinal adhesions have been implicated as a major contributing factor in failure of spinal surgery. Adhesion formation can be prevented by the application of a temporary biodegradable barrier between the tissues during wound healing thereby preventing or minimizing the presence of fibrous scar like tissue. Various biomaterials have been applied as a membrane, film, solution or gel to prevent post-surgical adhesions. U.S. Pat. No. 5,759,584 describes anti-adhesion devices based on poly trimethyl carbonate. Patent WO0167987 describes the use of polylactide membranes to prevent scar formation. Other applied materials include, polytetrafluorethylene, collagen, gelatin, oxidized regenerated cellulose, hyaluronic acid and carboxymethyl cellulose. Clinical successes in the prevention of fibrous tissue formation, however, have been limited. Advantages for the use thermogels as anti-adhesion barriers include its safe and complete bioresorbtion, high contact surface area, bio-adhesiveness, ease of usage by injection or spraying and low hydrogel swelling.

In another application, thermogels with LCST below 37° C. are suitable as a bulking agent to prevent or reduce stress urinary incontinence (SUI). SUI is a common and troublesome symptom amongst adult women. The periurethral or transurethral injection of bulking agents to increase urethral to intra-abdominal pressure by the induction of fibrous tissue is frequently applied. A review of applied bulking agents is provided by Kerr et al. “Bulking agents in the treatment of stress urinary incontinence: history, outcomes, patient populations, and reimbursement profile” Rev Urol, 7 (2005) Suppl 1, 3-11. Used materials include collagen, hyaluronic acid, silicone micro particles, hydroxyapatite, ethylene vinyl copolymers and polyacryl amide hydrogels. Durable improvements in urinary incontinence, however, are still limited. Reported issues include rapid decrease in tissue volume, particle migration to distant organs, lack of appropriate biocompatibility, granuloma formation, embolization and chronic inflammation. Thermogels are particularly suitable as a bulking agent since multiple injections are usually required, and thermogels are completely bioresorbable, biocompatible and non-tissue irritants. The thermogels are also suitable to be combined with synthetic or natural microparticles such as polylactide poly(lactic-co-glycolic acid), polycaprolactone, poly trimethylcarbonate, collagen or the like to further increase the fibrous tissue inducing capacity. The fast in situ gelling of thermogels ensures appropriate localized containment of microparticles thus preventing its migration.

In yet another useful application the thermogels with LCST below 37° C. can be used in ocular iontophoresis. Iontophoresis is a non-invasive drug delivery technique in which a small electric current is applied to enhance the penetration of ionized drugs into tissues. A review of this ophthalmic delivery approach is provided by Eljarrat-Binstock et al. “Iontophoresis: a non-invasive ocular drug delivery” J Control Rel, 110 (2006) 479-489. The method may use hydrogel sponges based on, for example, polyacetal or agar, which are saturated with the drug containing solution prior to use and placed directly onto the eye. Beneficial aspects for the use of thermogels in these applications include the high tissue contact area, high drug loading efficiency and bioavailability, ease of use and full biocompatibility with the ocular environment as well as its complete resorbtion.

In another suitable application the thermogel with LCST below 37° C., combined with a therapeutically active agent, can also be combined with imaging agents to monitor drug pharmacology including pharmocokinetics and pharmacodynamics. Diagnostic imaging techniques are reviewed by Saleem et al. “In vivo monitoring of drugs using radiotracer techniques” Adv Drug Del Rev, 41 (2000) 21-39, and can be performed by gamma scintigraphy including positron emission tomography, magnetic resonance imaging (MRI) and computed tomography (CT). Examples of suitable metal ions include for example Barium, Gadolinium, Manganese, Dysprosium, Europium, Lanthanum and Ytterbium. Examples of suitable radiolabels include Iodine, Carbon, Fluorine, Indium, Technecium and Cobalt. Microspheres containing water or air can also be used for MRI purposes. Advantages for the use of thermogels include their radiolucency and biocompatibility.

In still another application the thermogel with LCST below 37° C. can be used for reversible vessel occlusion. Vascular occlusion is a minimally invasive procedure intended to occlude blood vessels to protect a normal vascular bed, redirect the blood flow to the targeted site, and for conditions such as haemorrhage, vascular lesions, gastrointestinal bleeding or aneurysms. The embolic material can be introduced via a catheter or direct injection and requires radiopacity for visualization. In addition, the thermogel can also be injected for the occlusion of epicardial coronary arteries during off-pump coronary artery bypass to facilitate a bloodless field for optimal visibility during performance of the anastomosis. If required, the occlusion can be cleared by the addition of cold saline. Advantages for the use of thermogels for vessel occlusion include its fast gelling properties upon contact with body temperature, its thermoreversability, its biocompatibility, complete bioresorption without inducing adverse cellular events, and its hydrogel like properties which prevent mechanical injury to the endothelium as is the case with vascular clamps or shunts.

The invention is also useful to consistently deliver and dose so-called performance enhancing compounds to increase performance of tasks and assignments under prolonged stressful conditions. By performance enhancing compounds people skilled in the art refer to any set of molecules, extracts and formulations of synthetic or natural origin that can positively influence the physiological and psychological performance of humans with the objective to perform specific tasks and assignments under prolonged stressful or highly demanding conditions. Such performance enhancing compounds are for instance, but not limited to, painkillers, vitamins, caffeine-derivatives, antibiotics, anti-oxidants, extracts from plants, anabolic compounds, metabolic compounds, vasco-dilators and nutraceuticals. Examples of such stressful conditions or highly demanding conditions are for instance, but not limited to, combat activities, long-distance flights, long-distance sailing, professional deep-sea fishing and underwater welding, where fatigue, anxiety, physical and mental stress and loss of concentration can become detrimental to the completion of the task, or even dangerous to the individual and the team performing the tasks. These effects can be reduced, or the onset thereof delayed, by using above mentioned performance enhancing compounds and formulations.

The invention offers the benefit of increasing the compliance of the individual in the correct administration of the compounds or formulations, where regular and consistent administration is not a given or difficult to plan. The thermogel containing said performance enhancing compounds can be administered for instance by using injections under the skin or intra-muscular prior to commencing the activities. The performance enhancing compounds will slowly be released from the thermogel without the individual having to take any action or needing to think about taking a regular dose. Even more beneficial is that periodic repetition of the administration may be done, without scar formation or bio-accumulation of the polymer compounds or its degradation products.

The invention also relates to the use of compositions including the copolymers from the present invention, which are able to form thermogels in water with a LCST below 37° C. for the manufacture of a medicament. Such medicaments can be used in different applications like for example for use in tumor targeting, for use in the prevention of post-surgical adhesions, for use as a bulking agent for the prevention of stress urinary continence, for use in inflamed tissues, for use in ocular iontophoresis and ocular intravitreal injections, for use in temporary vessel occlusion, for the delivery of performance enhancing compounds under prolonged stressful or demanding conditions, for use in tissue engineering applications or for use as temporary in vivo void fillers.

The bulking agent can comprise synthetic or natural microparticles. Performance enhancing compounds may include compounds like a painkiller, a vitamin, a caffeine derivative, an antibiotic, an anti-oxidant, an extract from a plant, an anabolic, a metabolic, a vasco-dilators, a nutraceutical or combinations thereof.

The tissue engineering application can related to bone, cartilage, skin, nerve, muscle, ophthalmic and intervertebral disc repairs.

The thermogels can be combined with for example nerve-derived cells, chondrocytes, osteoblasts, bone marrow derived stem cells, mesenchymal stem cells, kidney derived cells, muscle derived cells, fibroblasts, keratinocytes, epithelial cells, fatty tissues derived cells, nucleus pulposus cells, annulus fibrosus cells or combinations thereof.

The compositions may also be sued as temporary void fillers for aesthetic surgery, reconstruction surgery and dental surgery.

The thermogels may be combined with for example synthetic or natural polymers. Such synthetic or natural polymers can be present as micro- or nanoparticles, microspheres, powders, fibres, fleeces, membranes, films or combinations thereof.

The compositions can be used for applications where a swelling of less than 5% in volume is of benefit, such as intra-ocular or intra-cranial surgery.

The therapeutically agent can be a growth factor, like for example one of the group existing of transforming growth factor beta-3, osteogenic protein 1, bone morphogenic proteins 2 and 7.

The invention is further explained in the experimental part, without being limited to the examples.

### Example 1

### Synthesis of an Amino-Terminated Polyacetal

The reaction was carried out in a glove box. 2.5 g (0.013 mol) 1,4-cyclohexanedimethanol divinyl ether, 1.6 g (0.011 mol) trans-1,4-cyclohexanedimethanol, and 0.23 g (0.82 mmol) of Fmoc-aminoethanol were dissolved in 10 ml of dry tetrahydrofuran. 0.2 ml of the catalyst, p-toluenesulfonic acid (10 mg/ml in tetrahydrofuran) was added under stirring and the reaction was carried out at room temperature for 5 h. Then 2.0 ml piperidine was added and the solution was stirred for another 2 h at room temperature. The product was purified by dialysis in tetrahydrofuran (molecular weight cut-off: 1000 Da) for 3 days and isolated by evaporation of the solvent. The prepolymer was characterized by ¹H NMR (CDCl₃) and GPC chromatography (in tetrahydrofuran, with polystyrene standards). The number average molecular weight according to GPC was 8000 Da

Final weight: ˜2.4 g

### Example 2

### Synthesis of an Amino-Terminated Poly (Ortho Ester)

The reaction was carried out in a glove box. 2.0 g (0.0094 mol) DETOSU, 1.24 g (0.0085 mol) trans-1,4-cyclohexanedimethanol, and 0.18 g Fmoc-aminoethanol were dissolved in 20 ml tetrahydrofuran. Two drops of the catalyst, p-toluenesulfonic acid (10 mg/ml in tetrahydrofuran) were added under stirring and the reaction was carried out at room temperature for 2 h. Then 4.0 ml piperidine was added and the solution is stirred for another 2 h at room temperature. The product was purified by dialysis in tetrahydrofuran (molecular weight cut-off: 1000 Da) for 3 days and isolated by evaporation of the solvent. The final weight was 2.3 grams. The product was characterised by ¹H NMR (CDCl₃) and GPC chromatography (in tetrahydrofuran, with polystyrene standards). The molecular weight was 10.000 Da.

### Example 3

### Synthesis of an Amino-Terminated poly [N-(2-hydroxyethyl)-L-glutamine]

2.0 g N-carboxyanhydride of γ-trichloroethyl-L-glutamate (TCEG-NCA) was dissolved in 20 ml dry 1,2-dichloroethane and the resulting solution was cooled down to 10° C. 0.099 g 1-triphenylmethylaminoethylamine (i.e. 5 mole % with respect to TCEG-NCA) was dissolved in 2 ml 1,2-dichloroethane and added to the solution of TCEG-NCA. Polymerization of TCEG-NCA proceeded by maintaining the temperature at 10° C. and was complete after 2 h (determined by infrared spectroscopy). Then a three-fold molar excess of acetic anhydride and equimolar quantity of triethylamine was added and the reaction mixture was stirred for 2 h at room temperature. The solution was precipitated in pentane and the polymer produced was isolated by filtration and dried under vacuum. The yield was 1.9 gram. Its molecular weight was determined by ¹H NMR (DMF-d7) and gel permeation chromatography (in tetrahydrofuran, with polystyrene standard): ˜6000 Da.

1.0 g (3.8 mmol) of the polymer obtained above was dissolved in 10 ml dry N,N-dimethylformamide. This solution was cooled down to 10° C. and 0.69 ml (11.5 mmol) ethanolamine and 0.36 g (3.8 mmol) 2-hydroxypyridine were then added. The reaction was followed by infrared spectroscopy and was complete after 2 h. The resulting aminolysed polymer was isolated by precipitation in ether, filtered, dried under vacuum and then purified by gel filtration on Sephadex G-25 (water as eluent) and isolated by lyophilisation. The yield was 1.0 gram. The purified polymer was characterised by ¹H NMR (D₂O) and gel permeation chromatography (in water, with dextran standards): 5000 Da.

1.0 g of the aminolysed polymer was dissolved in 10 ml trifluoroacetic acid and stirred at room temperature for 30 min. Trifluoroacetic acid was then removed by evaporation under vacuum. The resulting polymer was dissolved in water and centrifugated, then the supernatant was purified by GPC (Sephadex G-25, water as eluent; 5000 Da) and isolated by lyophilisation. Yield 0.9 g.

### Example 4

### Synthesis of a poly [N-(2-hydroxyethyl)-L-glutamine]-polyacetal diblock copolymer (PHEG-PA)

2.0 g 1,4-butanediol divinyl ether and 0.11 g Fmoc-aminoethanol were dissolved in 6 ml of dry tetrahydrofuran. 0.1 ml of the catalyst, p-toluenesulfonic acid (10 mg/ml in tetrahydrofuran) was added under stirring and the reaction was carried out at room temperature for 5 h. Then 1.2 ml piperidine was added and the solution was stirred for 2 h at room temperature. The product was purified by dialysis in tetrahydrofuran (molecular weight cut-off: 1000 Da) for 3 days and isolated by evaporation of the solvent. The product was characterised by ¹H NMR (CDCl₃) and GPC chromatography (in tetrahydrofuran, with polystyrene standards).

2.6 g N-carboxyanhydride of trichloroethyl-L-glutamate were dissolved in 10 ml dry chloroform. 1.5 g prepolymer was dissolved in 5 ml dry chloroform and added to the solution. The reaction was followed by infrared spectroscopy and was complete after 3 h mixing. 0.4 ml acetic anhydride and 0.6 ml triethylamine were added and the stirring continued for 2 h. At the end the product was precipitated in pentane, filtered, and dried under vacuum.

3.5 g copolymer were dissolved in dry N,N-dimethylformamide and cooled to 10° C. 1.7 ml 2-aminoethanol and 0.8 g 2-hydroxypyridine were added and the solution was stirred for 2 h. The reaction was followed by infrared spectroscopy. At the end the solvent was evaporated, the product was dissolved in water and purified by preparative GPC (Sephadex G-25, water as eluent). The final copolymer was isolated by lyophilisation.

Final weight: ˜1.5 g (first step), GPC in THF: ˜11 kDa
Final weight: ˜3.5 g (second step), GPC in THF: ˜19 kDa
Final weight: ˜3.0 g (third step), GPC in water: ˜16 kDa

### Example 5

### Synthesis of a poly[N-(2-hydroxyethyl)-L-glutamine]-poly(ortho ester)-poly[N-(2-hydroxyethyl)-L-glutamine] triblock copolymer (PHEG-POE-PHEG)

3.0 g of the prepolymer from example 2 were dissolved in 10 ml dry chloroform. 5.3 g N-carboxyanhydride of trichloroethyl-L-glutamate were dissolved in 50 ml dry chloroform and added to the solution. The mixture was stirred for 2 h. At the end of the reaction which was followed by infrared spectroscopy, 1.7 ml acetic anhydride and 2.2 ml triethylamine were added and the stirring continued for 2 h. At the end, the product was precipitated in hexane, filtered, and dried under vacuum.

7.0 g of the copolymer were dissolved in 70 ml dry N,N-dimethylformamide and cooled to 10° C. 3.6 ml 2-aminoethanol and 1.7 g 2-hydroxypyridine were added and the solution was stirred for 2 h. The reaction was followed by infrared spectroscopy. At the end the solvent was evaporated, the product was dissolved in water and purified by preparative GPC (Sephadex G-25, water as eluent). The final copolymer was isolated by lyophilization. Ultra-filtration using membranes with a defined molecular weight cut-off (from 1 kDa to 5 kDa depending on the expected polymer molecular weight) was an alternative to preparative GPC.

Final weight: ˜7.0 g (first step), GPC in THF: ˜28 kDa
Final weight: ˜3.5 g (second step), GPC in water: ˜24 kDa

### Example 6

### Synthesis of a polyacetal-poly[N-(2-hydroxyethyl)-L-glutamine]-polyacetal triblock copolymer (PA-PHEG-PA)

2.5 g (0.013 mol) 1,4-cyclohexanedimethanol divinyl ether and 1.9 g (0.013 mol) trans-1,4-cyclohexanedimethanol were dissolved in 10 ml of dry tetrahydrofuran. 0.2 ml of the catalyst, p-toluenesulfonic acid (10 mg/ml in tetrahydrofuran) was added under stirring and the reaction was carried out at room temperature for 5 h. Then 2 ml piperidine was added and the solution was stirred for another 2 h at room temperature. The product was purified by dialysis in tetrahydrofuran (MWCO 1000) for 3 days and isolated by evaporation of the solvent. The PA prepolymer was characterised by ¹H NMR (CDCl₃) and GPC chromatography (in tetrahydrofuran, with polystyrene standards).

4.4 g (0.013 mol) PA prepolymer and 13.3 g (0.052 mol) N,N′-disuccinimidyl carbonate were dissolved in 60 mL dry tetrahydrofuran. 9.1 mL (0.052 mol) diisopropylethylamine were mixed with 10 mL dry tetrahydrofuran and added slowly to the prepolymer solution at room temperature. After 8 h, the succinimidyl-modified PA prepolymer was purified by dialysis in tetrahydrofuran (MWCO 1000) for 3 days and isolated by evaporation of the solvent. The modified PA prepolymer was characterised by ¹H NMR (CDCl₃) and GPC chromatography (in tetrahydrofuran, with polystyrene standards).

5.2 g (0.013 mol) modified PA prepolymer and 2.6 g (6.5 mmol) amino-terminated poly[N-(2-hydroxyethyl)-L-glutamine] from example 3 were dissolved in 100 mL of 10 mM phosphate buffered saline (PBS) at pH 7.4. After 24 h of stirring at room temperature, the final PA-PHEG-PA copolymer was dialyzed to remove the PBS buffer (molecular weight cut-off: 1000 Da) and purified by subsequent preparative GPC (Sephadex G-25, water as eluent). The final PA-PHEG-PA copolymer was recovered by lyophilization.

Final weight: ˜4.4 g (first step), GPC in THF: ˜15 kDa
Final weight: ˜5.2 g (second step), GPC in THF: ˜16 kDa
Final weight: ˜7.0 g (third step), GPC in water: ˜36 kDa

### Example 7

### Synthesis of a poly(ortho ester) with pendant [N-(2-hydroxyethyl)-L-glutamine] groups (POE˜PHEGs)

1.0 g (0.0047 mol) 3,9-diethylidene-2,4,8,10-tetraoxospiro[5.5]undecane (DETOSU), 0.47 g (3.3 mmol) trans-1,4-cyclohexanedimethanol and 0.44 g (1.4 mmol) Fmoc-serinol were dissolved in 6 ml tetrahydrofuran. One drop of the catalyst, p-toluenesulfonic acid (10 mg/ml in tetrahydrofuran) was added under stirring and the reaction was carried out at room temperature for 2 h. Then 1.2 ml piperidine was added and the solution was stirred for 2 h at room temperature. The product was purified by dialysis in tetrahydrofuran (molecular weight cut-off: 1000 Da) for 3 days and isolated by evaporation of the solvent. The product was characterized by ¹H NMR (CDCl₃) and GPC chromatography (in tetrahydrofuran, with polystyrene standards).

1.5 g of the prepolymer was dissolved in 10 ml dry chloroform. 4.7 g N-carboxyanhydride of trichloroethyl-L-glutamate were dissolved in 50 ml dry chloroform and added to the solution. The mixture was stirred for 3 h. At the end of the reaction which is followed by infrared spectroscopy, 2.4 ml acetic anhydride and 3.0 ml triethylamine were added and the stirring continued for 2 h. At the end, the product was precipitated in hexane, filtered, and dried under vacuum.

4.0 g of the copolymer were dissolved in dry N,N-dimethylformamide and cooled to 10° C. 2.0 ml 2-aminoethanol and 0.9 g 2-hydroxypyridine were added and the solution was stirred for 2 h. The reaction was followed by infrared spectroscopy. At the end the solvent was evaporated, the product was dissolved in water and purified by preparative GPC (Sephadex G-25, water as eluent). The final copolymer was isolated by lyophilization.

Final weight: ˜1.5 g (first step), GPC in THF: ˜18 kDa
Final weight: ˜4.0 g (second step), GPC in THF: ˜40 kDa
Final weight: ˜3.6 g (third step), GPC in water: ˜36 kDa

### Example 8

### Determination of a Lower Critical Solution Temperature (LCST)

LCST properties (loss modulus G′, storage modulus G″, and complex viscosity η of the copolymers as a function of temperature) were determined by rheology (oscillation mode) using a Physica MC 301 (Anton Paar) rheometer.

Rheological properties at increasing temperatures were determined using the same polymer concentration as that used in gelling experiments, usually 20 wt %. FIG. 1 is a plot of the viscosity (y-axis, in mPa·s) versus temperature (x-axis, in ° C.). Although rheological measurements actually determined the onset of gelation shown as an increase of viscosity as a function of temperature, we defined the LCST as the temperature at which the viscosity started to increase for the compositions of this invention. In the example of FIG. 1, the LCST was 29° C.

### Example 9

### In Vitro Degradation Test of Thermogels

The degradation experiments were carried out in 10 mm diameter glass tubes with volume markings. The copolymer was dissolved at 20° C. in 10 mM phosphate buffered saline (PBS) at pH 7.4, and at a 20 wt % concentration, or in a 10 mM citric buffer at pH 5.5. 3.0 mL of solution were poured into each tube to ensure a solid gelation. The glass tubes were placed in an incubator with a shaking bath at 37° C. or in a water-bath thermostated at 37° C. for 1 h to make the 3 mL solutions gel. Then, 7.0 ml of 10 mM PBS at pH 7.4 or 7.0 ml of a citric buffer to pH 5.5 incubated at the same temperature were placed over the gels. At predetermined time periods, the buffer over the gel was withdrawn and the remaining volume of gel was measured through the volume marking. Then 7.0 mL of fresh buffer pre-incubated at the same temperature were added and the tubes were placed again into the thermostated bath. The remaining gel volumes were plotted against incubation time to get the degradation profiles.

### Example 10

### Preparation of Paclitaxel-Loaded Micelles

Some PHEG-POE-PHEG copolymer and Paclitaxel (1:0.4 w/w) were dissolved in acetonitrile and thoroughly mixed. The solvent was evaporated using a stream of nitrogen under stirring. The mixture was re-dissolved in distilled water and a solution with strong opalescence was obtained. After filtration (G3 filter), the solution was lyophilized. Micelles containing Paclitaxel could be smoothly re-dissolved in water and characterized by light-scattering measurements.

### Example 11

### In Vitro Release of Bovine Serum Albumine (BSA) from a Thermogel Followed by UV-Visible Light Spectroscopy

The release experiments were carried out in 10 mm diameter glass tubes. The copolymer was dissolved at 20° C. in 10 mM phosphate buffered saline (PBS) at pH 7.4, and at a 20 wt % concentration, or in a 10 mM citric buffer at pH 5.5. BSA at a loading of 1 wt % and 5 wt % was dissolved in the same buffer and mixed with the copolymer solution.

The glass tubes were placed in an incubator with a shaking bath at 37° C. or in a water-bath thermostated at 37° C. for 1 hour. The dimensions of the gel were 20 mm×10 mm. Then, 2 ml of 10 mM PBS at pH 7.4 or 2 ml of a citric buffer to pH 5.5 incubated at the same temperature were placed over the gels. At predetermined time periods, the buffer over the gel was withdrawn and replaced with a fresh buffer pre-incubated at the same temperature. The withdrawn samples were analyzed by UV-visible light spectroscopy using the absorption at 494 nm for pH 7.4 and the absorption at 458 nm for pH 5.5.

### Example 12

### Use of Thermogels as Temporary Void Filler and Shock Absorber in a Maxillo-Facial Trauma

Upon arrival of a patient to the emergency ward, and after diagnosis of a significant maxillo-facial trauma, a biodegradable thermogel would be injected in the damage areas in order to relieve pain (via an analgesic contained in the composition) and act as a shock absorber between broken bone and tissue parts upon gelation. The gel would also prevent unwanted adhesion of damaged tissue and bones to prevent scar tissue formation. This would give the surgeons more time to plan reconstructive surgery and would cause less trauma for the patient during reconstructive surgery because spontaneous healing would delayed for a few days. By the time the surgeons would be ready, the gel would have started degrading or remaining gel blocks could be removed by cooling them down using cold fluids or instruments and then by sucking the liquefied gel out.

### Examples 13

### Creation of a Nerve Guide With an Inner Lining Containing Growth Factors and a Lumen Containing Nerve Stem Cells

A tube with an internal diameter matching the external diameter of the nerve to repair was dip-coated on the inside with a cold composition containing an amphiphilic copolymer from this invention (giving a LCST of 15° C.), nutrients for cell growth and growth factors. After draining off the excess of composition material, the tube temperature was raised to 20° C. to gel the composition as an inner lining against the tube wall. Then the tube lumen was filled by dip-coating with a solution containing nerve stem cells and an amphiphilic copolymer from this invention (giving a LCST of 25° C.). The tube temperature was raised to 30° C. to gel the solution in the lumen. This tube was implanted at the severed nerve location. Nerve re-growth could occur through rapid erosion of the lumen gel to expose nerve stem cells to the extremities of the severed nerve, and the growth would be sustained by the outer gel coating supplying nutrients and growth factors to the lumen at an optimal rate.

### Example 14

### Injection of a Thermogel Containing Osteogenic and/or Bone Morphogenic Proteins into Intervertebral Discs or Articulate Cartilage to Stop or Reverse Degeneration of Diseased or Damaged Tissues

A composition of the thermogel with a LCST of 37° C. containing amongst other components the growth factor TGF-beta-3, or another osteogenic or bone morphogenic protein was prepared. The composition in its liquid form was injected into the intervertebral disc using a small bore needle or a small diameter cannula. Upon reaching LCST, the composition would gel and hold the growth factor in situ over a period of time, releasing it in a slower manner than straight injection of a non-gelling solution.

1. Amphiphilic copolymer, containing at least a hydrophilic chain segment (A) and a hydrophobic chain segment (B), wherein the hydrophilic chain segment (A) contains peptides and wherein the hydrophobic chain segment (B) contains acetal groups or orthoester groups.

2. The copolymer according to claim 1, wherein the hydrophilic chain segment (A) contains glutamine/glutamic acid units or asparagines/aspartic acid units.

3. Copolymer according to claim 1, characterized in that the copolymer is a block copolymer or a graft copolymer.

4. Copolymer according to claim 1, characterized in that the copolymer is an A(BA)ₓ, B(AB)ₓ or (AB)ₓ block copolymer, wherein x is an integer between 1 and 5.

5. Copolymer according to claim 1, characterized in that the hydrophilic chain segment(s) contains units of N-hydroxyalkyl-L-glutamine, N-alkyl-L-glutamine, L-glutamic acid, N-hydroxyalkyl-L-aspartamine, N-alkyl-L-aspartamine, L-aspartic acid or a combination thereof.

6. Copolymer according to claim 1, characterized in that the hydrophilic chain segment(s) is poly[N-hydroxyalkyl-L-glutamine], poly[N-alkyl-L-glutamine], poly[L-glutamic acid], poly[N-hydroxyalkyl-L-aspartamine], poly[N-alkyl-L-aspartamine], poly[L-aspartic acid].

7. Copolymer according to claim 1, characterized in that the hydrophobic chain segment(s) contains poly-L-lactide, poly-D,L-lactide, poly(lactide-co-glycolide), polyanhydride, polyphosphazene, polyketals.

8. Copolymers according to claim 6, characterized in that copolymer is one out of the group poly[N-hydroxyalkyl-L-glutamine]-polyacetal, poly[N-hydroxyalkyl-L-glutamine]-polyacetal-poly[N-hydroxyalkyl-L-glutamine], polyacetal-poly[N-hydroxyalkyl-L-glutamine]-polyacetal block copolymers, polyacetal-poly[N-hydroxyalkyl-L-glutamine] graft copolymers, poly[N-hydroxyalkyl-L-glutamine]-poly(ortho ester), poly[N-hydroxyalkyl-L-glutamine]-poly(ortho ester)-poly[N-hydroxyalkyl-L-glutamine], poly(ortho ester)-poly[N-hydroxyalkyl-L-glutamine]-poly(ortho ester) block copolymers, poly(ortho ester)-poly[N-hydroxyalkyl-L-glutamine] graft copolymers, poly[N-hydroxyalkyl-L-aspartamine]-polyacetal, poly[N-hydroxyalkyl-L-aspartamine]-polyacetal-poly[N-hydroxyalkyl-L-aspartamine], polyacetal-poly[N-hydroxyalkyl-L-aspartamine]-polyacetal block copolymers, polyacetal-poly[N-hydroxyalkyl-L-aspartamine] graft copolymers, poly[N-hydroxyalkyl-L-aspartamine]-poly(ortho ester), poly[N-hydroxyalkyl-L-aspartamine]-poly(ortho ester)-poly[N-hydroxyalkyl-L-aspartamine], poly(ortho ester)-poly[N-hydroxyalkyl-L-aspartamine]-poly(ortho ester) block copolymers, poly(ortho ester)-poly[N-hydroxyalkyl-L-aspartamine] graft copolymers, poly[N-alkyl-L-glutamine]-polyacetal, poly[N-alkyl-L-glutamine]-polyacetal-poly[N-alkyl-L-glutamine], polyacetal-poly[N-alkyl-L-glutamine]-polyacetal block copolymers, polyacetal-poly[N-alkyl-L-glutamine] graft copolymers, poly[N-alkyl-L-glutamine]-poly(ortho ester), poly[N-alkyl-L-glutamine]-poly(ortho ester)-poly[N-alkyl-L-glutamine], poly(ortho ester)-poly[N-alkyl-L-glutamine]-poly(ortho ester) block copolymers, poly(ortho ester)-poly[N-alkyl-L-glutamine] graft copolymers, poly[N-alkyl-L-aspartamine]-polyacetal, poly[N-alkyl-L-aspartamine]-polyacetal-poly[N-alkyl-L-aspartamine], polyacetal-poly[N-alkyl-L-aspartamine]-polyacetal block copolymers, polyacetal-poly[N-alkyl-L-aspartamine] graft copolymers, poly[N-alkyl-L-aspartamine]-poly(ortho ester), poly[N-alkyl-L-aspartamine]-poly(ortho ester)-poly[N-alkyl-L-aspartamine], poly(ortho ester)-poly[N-alkyl-L-aspartamine]-poly(ortho ester) block copolymers, poly(ortho ester)-poly[N-alkyl-L-aspartamine] graft copolymers, poly(L-glutamic acid)-polyacetal, poly(L-glutamic acid)-polyacetal-poly(L-glutamic acid), polyacetal-poly(L-glutamic acid)-polyacetal block copolymers, polyacetal-poly(L-glutamic acid) graft copolymers, poly(L-glutamic acid)-poly(ortho ester), poly(L-glutamic acid)-poly(ortho ester)-poly(L-glutamic acid), poly(ortho ester)-poly(L-glutamic acid)-poly(ortho ester) block copolymers, poly(ortho ester)-poly(L-glutamic acid) graft copolymers, poly(L-aspartic acid)-polyacetal, poly(L-aspartic acid)-polyacetal-poly(L-aspartic acid), polyacetal-poly(L-aspartic acid)-polyacetal block copolymers, polyacetal-poly(L-aspartic acid) graft copolymers, poly(L-aspartic acid)-poly(ortho ester), poly(L-aspartic acid)-poly(ortho ester)-poly(L-aspartic acid), poly(ortho ester)-poly(L-aspartic acid)-poly(ortho ester) block copolymers, poly(ortho ester)-poly(L-aspartic acid) graft copolymers.

9. Copolymers according to claim 1, containing moieties that allow chemical reactions to occur between the polymer chains to achieve polymer crosslinking.

10. Copolymers according to claim 1 and with a structure compliant to formula I 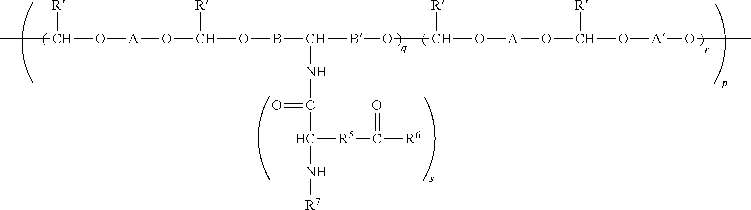
where: R′ is a C₁ to C₄ alkyl chain.
s=integer from 2 to 20
q and r are independent integers between 1 and 20
p is an integer between 3 and 30
B and B′ are C₁ to C₅ alkyl chains.
A and A′ can be R², R³, or a mixture thereof.
R² is selected from: 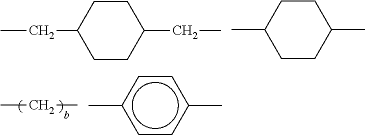
where b is an integer between 1 and 12.
R³ is: 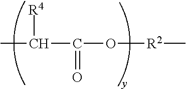
where R⁴ is H or a C₁ to C₆ alkyl chain and y is an integer between 1 and 10.
R⁵═(CH₂)_{z}
z is integer between 1 and 6.
R⁶═OH, OCH₃, NH—(CH₂—)ₙOH, N—(CH₂—CH₂—OH)₂, NH—(CH₂—CH₂—O—)_{z}H, NH—CH₂—CH(OH)—CH₂—OH, NH—(CH₂—), O—CO—CH═CH₂, NH—(CH₂—)_{z}O—CO—C(CH₃)═CH₂, N—(CH₃)₂, N—CH—(CH₃)₂.
z is an integer between 1 and 6.
R⁷═CO—CH₃ CO—CH═CH₂, CO—C(CH₃)═CH₂.

11. Copolymers according to claim 1 and with a structure compliant to formula II 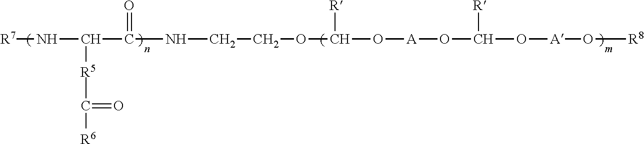
where A, A′, R′, R⁵, R⁶ and R⁷ are defined as stated in claim 10 (formula I);
n is an independent integer between 2 and 200;
m is an independent integer between 2 and 150;
R⁸═H, R⁷, R¹⁰, CH(R)—O—R¹⁰.
R¹⁰=C₁ to C₁₆ alkyl chain (linear or branched), cyclohexyl.

12. Copolymers according to claim 1, and with a structure compliant to formula III 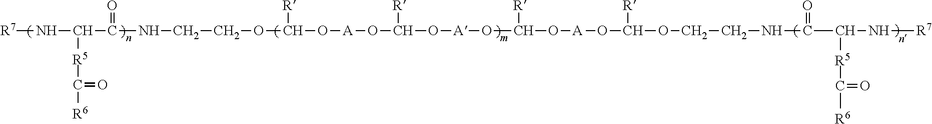
where A, A′, R′, R⁵, R⁶ and R⁷ are defined as stated in claim 10; n and n′ are an independent integers between 2 and 200; m is an independent integer between 2 and 150.

13. Copolymers according to claim 1, and with a structure compliant to formula IV 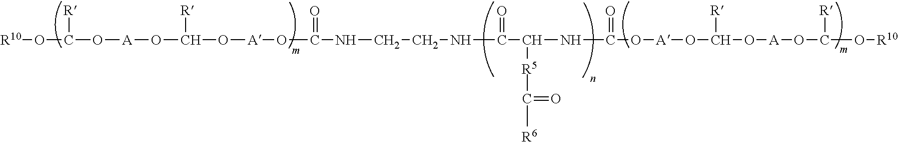
where A, A′, R′, R⁵, R⁶ and R¹⁰ are defined as stated in claim 10. n is an independent integers between 2 and 200; m is an independent integer between 2 and 150.

14. Copolymers according to claim 1, and with a structure compliant to formula V 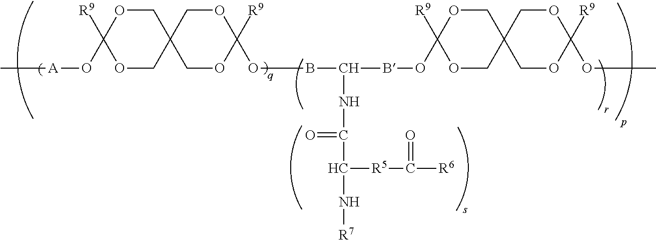
where A, B, B′, R⁵, R⁶ and R⁷ are defined as stated in claim 10; R⁹=—H, C₁ to C₄ alkyl chain.

15. Copolymers according to claim 1, and with a structure compliant to formula VI 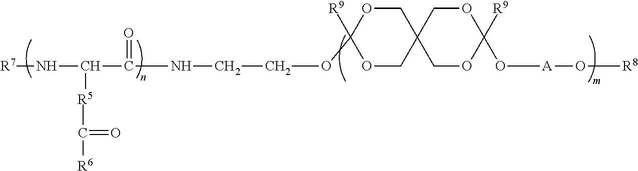
where A, R⁵, R⁶, R⁷ and Ware defined as stated in claim 10
R⁹ is defined as stated in formula V.
n is an independent integer between 2 and 200;
m is an independent integer between 2 and 150;

16. Copolymers according to claim 1, and with a structure compliant to formula VII 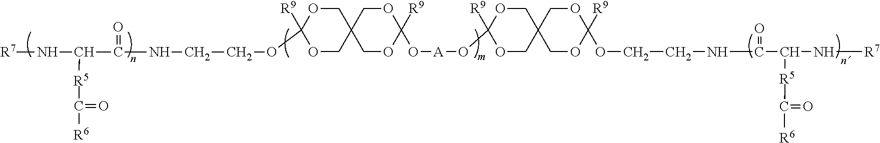
where A, R⁵, R⁶ and R⁷ are defined as stated in claim 10.
R⁹ is defined as stated in formula V.
n and n′ are an independent integers between 2 and 200;
m is an independent integer between 2 and 150

17. Copolymers according to claim 1, and with a structure compliant to formula VIII 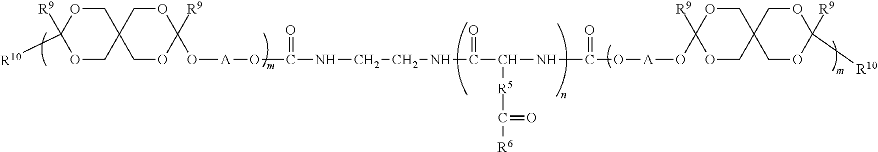
where A, R⁵, R⁶ and R¹⁰ are defined as stated in formula II.
R⁹ is defined as stated in formula V.
n is an independent integer between 2 and 200;
m is an independent integer between 2 and 150;

18. A composition containing at least one amphiphilic copolymer of claim 1, and at least one therapeutically active agent.

19. A composition according to claim 18, characterized in that the copolymer forms micelles in water, the therapeutically active agent being entrapped in the micelles.

20. A composition according to claim 18, characterized in that the copolymer forms polymersomes in water, the therapeutic agent being entrapped in the polymersomes, the therapeutic agent being, but not limited to haemoglobin to form artificial blood.

21. A composition according to claim 18, characterized in that the composition when put in water can form a thermogel with a LCST below 37° C.

22. An implant containing the copolymer according to claim 1.

23. An implant according to claim 22, wherein the implant contains at least one therapeutically active agent.

24. An implant according to claim 22, wherein the copolymer can form a thermogel with a LCST below 37° C. and is contained in the lumen of the implant.

25. Compositions including copolymers from claim 1, which are able to form thermogels in water with a LCST below 37° C. and containing at least one imaging agent for use in diagnostic imaging.

26. Use of compositions including copolymers from claim 1, which are able to form thermogels in water with a LCST below 37° C. for the manufacture of a medicament for use in tumor targeting, for use in the prevention of post-surgical adhesions, for use as a bulking agent for the prevention of stress urinary incontinence, for use in inflamed tissues, for use in ocular iontophoresis and ocular tissues, for the use in tissues of the nervous system and the brain, for use in temporary vessel occlusion, for the delivery of performance enhancing compounds under prolonged stressful or demanding conditions, for use in tissue engineering applications or for use as temporary in vivo void fillers.

27. Use of compositions according to claim 26 wherein the bulking agent comprises synthetic or natural microparticles.

28. Use of compositions of claim 26 wherein the performance enhancing compounds include a painkiller, a vitamin, a caffeine derivative, an antibiotic, an anti-oxidant, an extract from a plant, an anabolic, a metabolic, a vasco-dilator, a nutraceutical or combinations thereof.

29. Use of compositions according to claim 26 wherein the tissue engineering application is related to bone, cartilage, skin, nerve, muscle, ophthalmic, hormone secreting gland tissues and intervertebral disc repairs.

30. Use of compositions according to claim 26, wherein the thermogels are combined with nerve-derived cells, chondrocytes, osteoblasts, bone marrow derived stem cells, mesenchymal stem cells, kidney derived cells, muscle derived cells, fibroblasts, keratinocytes, epithelial cells, fatty tissues derived cells, nucleus pulposus cells, annulus fibrosus cells, embryonic stem cells or combinations thereof.

31. Use of the compositions of claim 26, as temporary void fillers for aesthetic surgery, reconstruction surgery and dental surgery.

32. Use of compositions of claim 31 wherein the thermogel is combined with synthetic or natural polymers.

33. Use of the compositions of claim 32 wherein the synthetic or natural polymers are present as micro- or nanoparticles, microspheres, powders, fibres, fleeces, membranes, films or combinations thereof.

34. Use of the compositions of claim 18, anyone for applications where a swelling of less than 15% in volume is of benefit, such as intra-ocular or intra-cranial surgery.

35. Compositions according to claim 18, where the therapeutically agent is a growth factor.

36. Composition according to claim 35, wherein the transforming growth factor is at least one of the group existing of transforming growth factor beta-3, osteogenic protein 1, bone morphogenic proteins 2 and 7.
